(19) United States
(12) Patent Application Publication
(10) Pub. No.: US 2011/0282189 A1
(43) Pub. Date: Nov. 17, 2011

(54) METHOD AND SYSTEM FOR DETERMINATION OF 3D POSITIONS AND ORIENTATIONS OF SURGICAL OBJECTS FROM 2D X-RAY IMAGES

(76) Inventor: Rainer Graumann, Hoechstadt (DE)

(21) Appl. No.: 13/106,102

(22) Filed: May 12, 2011

(30) Foreign Application Priority Data
May 12, 2010 (DE) 10 2010 020 284.3

(51) Int. Cl. A61B 6/00; G06K 9/00

(52) U.S. Cl. 600/427; 382/132

(57) ABSTRACT

In a method, a system and a computer readable storage medium encoded with programming instructions, as well as a calculation module for three-dimensional presentation of at least two separate surgical objects within a medical procedure, access to three-dimensional models for the surgical objects takes place based on an acquired two-dimensional x-ray image with the surgical objects. The accessed three-dimensional models are integrated into the acquired two-dimensional x-ray image in order to be shown as a modified x-ray image. The modified x-ray image includes position information, relative positions and orientations of the surgical objects.

### BACKGROUND OF THE INVENTION

1. Field of the Invention

The present invention is in the field of medical technology and information technology, and in particular concerns an approach to allow surgical objects (such as implants, tools and instruments) to be located with regard to their position and orientation in space for a medical procedure, based on monoplanar (two-dimensional) x-ray images.

2. Description of the Prior Art

In recent years the use of computer-assisted surgical systems (CAS) has significantly increased in the field of surgery, not the least because, with computer-based use, operation times can be shortened, preoperative operation preparations and operation results can be improved (and in particular can be designed more efficiently). Moreover, it an sometimes be necessary to interrupt the operation, for instance due to required imaging measures, and computer assistance allows a resumption of the operation at the precise point of interruption.

In the fields of orthopedics and traumatology, for example in operation on femur fractures or hip joints, as well as in the field of cardiac surgery, it can be necessary to introduce or correctly position implants or instruments in the patient under two-dimensional x-ray image monitoring. Normally, preoperative two-dimensional or three-dimensional x-ray image exposures of the appertaining anatomical structure are produced in order to be able to plan the operation flow.

The problem is now present that in principle no three-dimensional information can be derived from the intraoperatively acquired two-dimensional x-ray images. In particular, it is thus not possible to measure relative distances between an implant and a tool, for example. Furthermore, it is not possible to derive information about the orientation (among other things an angle relative to the anatomical structure) of a tool, for example a screw to be introduced.

In order to solve this problem, different methods are used in the prior art.

It is known to produce multiple x-ray images of the respective anatomical structure from different directions. For example, two orthogonal exposures are typically acquired in order to obtain additional three-dimensional information from these two exposures. A disadvantage of this method is that the variables (distances and orientations) cannot be quantitatively measured and accordingly can be visualized only to a limited extent, or even not at all.

The Surgix© system, which has been developed in order to assist in orthopedic and traumatological procedures using software and hardware components, is an example of an additional computer-assisted surgical system. This system is based on objects (for example instruments with x-ray-opaque markers) being provided so that the positions of the markers in the x-ray image are visible. In a subsequent post-processing step the position and orientation of the respective instrument in space (in particular in the coordinate system of the x-ray apparatus) can be calculated and visualized from the position of the marker.

### SUMMARY OF THE INVENTION

An object of the present invention is to provide a procedure to measure and exactly visualize by means of the one three-dimensional localization and orientation of surgical objects (for example a relative distance and angle specifications between an implant and a tool), wherein only a single x-ray exposure is necessary for this purpose.

In the following the achievement of the object is described using the method according to the invention. Advantages, features and/or alternative embodiments that are hereby mentioned are similarly also to be transferred to the other embodiments of the invention (system, calculation module and computer program) and vice versa. The functional features of the method are realized via corresponding hardware modules whose functionality coincides with that of the functional feature, and vice versa.

In the method according to the invention, surgical objects should be visualized in a three-dimensional representation. Surgical objects are all objects that are relevant within the scope of a medical procedure and include at least one implant, at least one surgical tool (for example a drill or a spacer) and at least one surgical instrument. These do not necessarily need to be objects that are used exclusively in an intraoperative field but also can be objects that are used alternatively or cumulatively in preoperative procedures, thus for example in operation preparation. A frequent application case concerns the localization of an implant and a surgical tool for processing the implant. It is important for the tool to be directed or brought to exactly the correct position and/or correct angle with regard to the implant for insertion (use). According to the invention, the tool can be directed to this correct position via software assistance. It should be emphasized that this is executed both as a preparatory measure (thus outside of the patient body; in this case the method is exclusively percutaneous), or the position determination can already take place in operative usage and thus at the patient during the procedure. In principle, the method according to the invention can also be used for more than two surgical objects and can be applied for these simultaneously in order to jointly visualize these in a virtual spatial presentation (for example an implant, a tool and an instrument).

Most application cases concern the use of at least one tool and/or one instrument with regard to a separate surgical object, for example an implant that has already been introduced into the body. The method is therefore related to the three-dimensional representation of at least two separate surgical objects. However, it is likewise within the scope of this invention to apply the method only to one surgical object.

The term “medical procedure” is to be broadly understood and includes all forms of a medical procedure, from minimally invasive procedures to complex operations. Medical monitoring examinations (for example to check a position of an implant) are likewise comprised. Moreover, the medical procedure can concern the field of treatment planning (and thus preoperative treatment steps) or even the operative use and the steps implemented during the operation.

A three-dimensional model for a particular surgical object is typically provided. The manufacturers of implants normally provide such a two-dimensional model. Otherwise, such a model can be calculated relative to other points or can also be newly calculated if necessary. The three-dimensional model is typically stored in a database (to which the method according to the invention has access). The three-dimensional model comprises all spatial coordinates of the subject and in particular its shape, size and dimensions.

In a preferred embodiment of the invention, a modified x-ray image is shown. However, the modification of the acquired x-ray image is not mandatory. Alternatively, it is also possible to show the three-dimensional position data (with the orientations) in a separate three-dimensional representation and, so to speak, to not integrate these into the x-ray image or superimpose these three-dimensional position data on said x-ray image. In this case the modified x-ray image merely consists of the calculated three-dimensional positions that are shown in a virtual space. According to the invention, the modified x-ray image differs from the acquired x-ray image in that it includes additional information with regard to localization and orientation of the surgical objects. It includes a virtual representation of surgical objects that can be integrated into the x-ray image, or be overlaid on the image as supplementary information (for example also in a different format, as in a table). The presentation of the modified x-ray image typically takes place on a monitor or, respectively, a screen of a connected computer.

An important aspect of the present invention is apparent in that a three-dimensional localization of medically relevant objects and structures in space (it can also be more than two surgical objects) is enabled, although only a two-dimensional x-ray image is provided. A marker, a three-dimensional model for the object or a marker and a model that are used for 3D position calculation are advantageously associated with each object.

The invention here provides two fundamental embodiments for the introduction of three-dimensional variables and information in the monoplanar x-ray image, which can also be combined.
1. It is possible to access a three-dimensional model of the relevant surgical objects in which spatial data are already stored. These spatial data are then introduced into or adapted to the two-dimensional x-ray image by selecting or determining a two-dimensional representation of the three-dimensional model, from a provided set of two-dimensional representations of the three-dimensional model, that has a maximum coverage or agreement when superimposed with the acquired two-dimensional x-ray image. In other words, a congruence image that coincides with the acquired x-ray image is selected from the set of two-dimensional representations of the three-dimensional model. The spatial data of this congruence image are then used for the three-dimensional representation of the modified x-ray image.
2. A second approach does not (like the first approach described above) necessarily require a three-dimensional model, but rather is based on the use of markers. The surgical objects that are relevant to the medical procedure are thereby provided with x-ray-opaque markers. The respective surgical objects together with their markers must be visible on the monoplanar x-ray image to be acquired. There is typically an unambiguous association between a respective marker system and a respective surgical object. The association can but does not need to be one-to-one. For example, it is conceivable that the markers are clipped to a surgical wire but it remains capable of rotating around the wire, depending on medical or other application. A marker system normally consists of at least 4 marker spheres that are arranged in a specific geometric pattern (for example, these 4 spheres do not need to lie in one plane). The markers can now have a specific geometric relationship to the surgical object. Three-dimensional information with regard to the surgical objects can be derived from the acquired x-ray image with the surgical objects and their markers. This three-dimensional information in particular relates to a position of the surgical objects, to relative positions or to a distance of the objects from one another and to orientations and angle specifications (comprising the angles in the three spatial axes) of the objects in space. For example, it is possible for a drill to be directed exactly in the correct orientation outside of the body to a drill sleeve that (for example) is located in an implant in the patient body. The angle of the instrument (drill) thus coincides with the angle of the respective implant structure into which the instrument should be introduced. This three-dimensional information can then be presented in the modified x-ray image just as in the first embodiment.

A combination of the first approach and the second approach is also within the scope of the invention as a third approach. In particular, the marker-based approach can also make use of a stored model. In this alternative embodiment of the invention, markers are attached in advance to the surgical structures whose position in the two-dimensional x-ray image is detected.

A 3D model for these surgical structures is additionally accessed. The modified x-ray image with all 3D information necessary for navigation is then calculated from both input data and presented.

In principle, it is merely necessary to acquire an x-ray image with the surgical objects before the procedure. A virtual presentation of the objects (in the virtual 3D space of the x-ray apparatus) is then possible based on the mathematical adaptation. Navigation can also take place in this presentation.

As soon as a position variation or a relative movement between the structures and/or objects results, a new x-ray exposure can be initiated—possibly automatically or after a confirmation signal of the user—in order to update the presentation with the respective positions and orientations. The method steps are then executed iteratively in order to achieve an updated presentation given a relative position variation.

The modified x-ray image comprises all three-dimensional information that are relevant to the medical procedure. In particular, the position information for the relevant medical objects (for example implant, tool and instrument) and their orientations in space are comprised here. In the preferred embodiment the term “three-dimensional position” should also comprise all relevant orientations and angle specifications regarding the respective objects. In an alternative embodiment, it is also possible to additionally show further additional information or to introduce it into the modified x-ray image, for example the overlaying of additional data. For example, these data can comprise warning notices in the event that the position of the tool or instrument has been calculated to be unsuitable or as not matching with regard to the reference subject (for example the implant). Moreover, additional metadata can be overlaid, for example the datum of the respective x-ray exposure, the manufacturer of the implant or additional data with regard to the patient, the medical procedure, the means used in the medical procedure etc. It is likewise possible to superimpose additional image data on the x-ray image or to introduce this into the x-ray image, for example as a miniature presentation or as a virtual presentation. For example, earlier exposures of the same anatomical structure (for example before a fracture) can be helpful.

It is likewise possible to fuse the modified x-ray image with an additional image in order to present additional information. For example, the additional image can be an image of a different modality (CT, MRT, PET, ultrasound etc.). Moreover, it is possible to overlay reference images that indicate to the operator how an optimal procedure between tool and implant has to appear. For example, here it can be shown how deep a screw should be driven into the implant, or in what position a k-wire should be positioned to temporarily fix an implant.

An additional important aspect of the present invention is that the method according to the invention serves as a basis for a navigation-guided medical procedure. As was already mentioned in the preceding, via the presentation of the modified x-ray image with the three-dimensional information it is possible to better plan the operation procedure in advance. In particular, the positioning of the tools and instruments can be targeted. In other words, it is possible that surgical instruments and tools can be directed in a dedicated manner to an (anatomical) goal. For example, if it is necessary to drive a screw (as a tool) into a thread, the screw must be guided to the correct spatial position and the middle longitudinal axis of the screw must additionally coincide with the middle longitudinal axis of the thread. This can be prepared before the screw is inserted into the anatomical structure. An additional example is distal pin locking. Here the position and direction of the pin is controlled via the modified x-ray image (which, among other things, can be generated from an image fusion with the model).

Moreover, in a further embodiment it is possible to already display preoperatively planned implant placements and access paths in advance of a medical procedure.

The method according to the invention is in principle not limited to the acquisition of only one x-ray image exposure. It has proven to be an important advantage that all relevant position information can be determined from only one x-ray image exposure (which lowers or, respectively, reduces the radiation exposure and the time duration for the treatment of the patient). Although it is naturally also possible to produce multiple x-ray images and to use these in the presentation, it is not necessary. For example, the additional x-ray exposures can be monitoring exposures that are superimposed on the modified x-ray image or overlaid in the modified x-ray image or can additionally be shown as a separate image. The method according to the invention can thus also be applied iteratively, both preoperatively and intraoperatively.

According to a preferred embodiment of the invention, the spatial position information is presented in a coordinate system of the C-arm of the acquiring x-ray apparatus. However, it is likewise within the scope of the invention to implement an (additional) coordinate transformation here in order to be able to visualize the relevant data in a different coordinate system.

In a preferred embodiment, the method according to the invention can be subdivided into two time phases:
1. An acquisition phase. The acquisition phase serves for the image acquisition of the two-dimensional x-ray image.
2. A visualization phase. The visualization phase serves for the calculation of the three-dimensional data and to present the modified x-ray image with the three-dimensional data of the surgical objects.

With regard to the flexibility of the method according to the invention it has particularly advantageously been proven that the acquisition phase is independent of the calculation and visualization phase. The single requirement is that an x-ray image must be acquired for the calculation and visualization phase. The exact time period is not necessarily predetermined. However, for practical reasons it is reasonable for the point in time for the acquisition of the x-ray image to optimally take place immediately before the medical procedure in order to avoid errors due to position variations.

The invention has a number of advantages.

With the invention it is possible to present spatial relationships of the participating tools, implants or additional surgical objects three-dimensionally in order to use the spatial information for the placement of the implants and the relevant tools using exclusively a monoplanar x-ray image.

This ultimately leads to shorter operating times, to a lower amount of x-ray radiation for the patient, an increase in quality or, respectively, safety in medical procedures, to reduce medical complications and ultimately to reduce invasive or operation procedures.

The invention also encompasses a system for three-dimensional presentation of at least two separate surgical objects within the scope of an imaging-assisted, medical procedure, having:
at least one imaging unit, in particular an x-ray apparatus with C-arm, that serves to acquire a two-dimensional x-ray image in which are jointly shown the at least two surgical objects;
optionally at least one database in which are stored three-dimensional models for the surgical objects;
at least one calculation model that is designed to calculate three-dimensional positions, comprising all position and angle information relevant to a navigation, from the at least two surgical objects in a coordinate system of the imaging unit, wherein the calculation is based on prepared three-dimensional models for the at least two surgical objects and/or on markers that are attached to the respective surgical objects for the purposes of the position determination or image registration and
at least one presentation module that is fashioned to present a modified x-ray image in a virtual 3D space and is based on the calculated positions of the calculation module, wherein position information and orientations between the at least two surgical objects can be measured from the three-dimensional model and/or from the modified x-ray image and are presented. The presentation module can additionally or cumulatively be fashioned in order to present the 3D positions and orientations of the at least two surgical objects in a virtual 3D space.

The units and modules cited in connection with the system can be software and/or hardware modules that are engaged in a data exchange with one another. The calculation module, the presentation module and the additional modules can be fashioned as structural units in a microprocessor. The data exchange typically takes place via a network or a bus system.

The invention also encompasses a calculation module for calculation of three-dimensional position information and orientations in space between at least two surgical objects within the framework of an imaging-assisted medical procedure, wherein the calculation module calculates a modified x-ray image from a provided two-dimensional x-ray image in which are jointly shown the at least two surgical objects and/or with access to marker data, wherein the marker data relate to spatial position information of markers that have been attached to the at least two surgical objects (in advance for the purposes of position determination) and are mutually visible in the acquired x-ray image.

In an embodiment the calculation module can additionally be fashioned to show the modified x-ray image, such that the operator has provided all relevant data for spatial navigation of the surgical object with regard to the implant. The modified x-ray image in particular comprises distance information between the tool or, respectively, instrument and the implant and its respective alignment in the coordinate system of the C-arm. The surgical object (tool or instrument) can therefore specifically be directed towards the implant, and furthermore it is possible to adapt its spatial orientation (example: attitude of the middle longitudinal axis of the “screw” object) to the spatial orientation of the implant (example: middle longitudinal axis of a “drill sleeve” in the implant).

The invention also encompasses a non-transitory computer-readable storage medium encoded with programming instructions. The programming instructions, when the storage medium is loaded into a processor supplied with the aforementioned two-dimensional x-ray image and the aforementioned three-dimensional model, cause the processor to implement the above-described method according to the invention, as well as all embodiments thereof.

As mentioned above, a primary goal of the present invention is to provide a modified x-ray image with relevant spatial coordinates of the surgical objects. In an alternative embodiment, a modified x-ray image is not shown but rather only the relevant position information between the surgical objects is shown (for example the relative position, the spatial attitude and its orientation). These data can be output in different formats. In addition to an optical format (that relates to the embodiments in the presentation of a modified x-ray image, which embodiments are described above), an acoustic format (for example) is also possible in the form of an acoustic warning notification given incorrect positioning. In particular, it is possible that the operator is informed via an acoustic signal in the event that the instrument or, respectively, tool is not located in the target position and target orientation and/or that a different acoustic signal is output in the event that the tool or instrument is located in the target position and/or target orientation.

### BRIEF DESCRIPTION OF THE DRAWINGS

FIG. 1 is a schematic overview presentation of a system according to the invention, according to a preferred embodiment.

FIG. 2 is a workflow diagram with two embodiment variants of the invention.

FIG. 3 is a schematic presentation of an embodiment of the method according to the invention in which a k-wire with markers is transformed into a three-dimensional space.

FIG. 4 is a schematic presentation of the method according to the invention according to a preferred embodiment, in the example of a distal pin locking and the presentation of a k-wire with marker.

### DESCRIPTION OF THE PREFERRED EMBODIMENTS

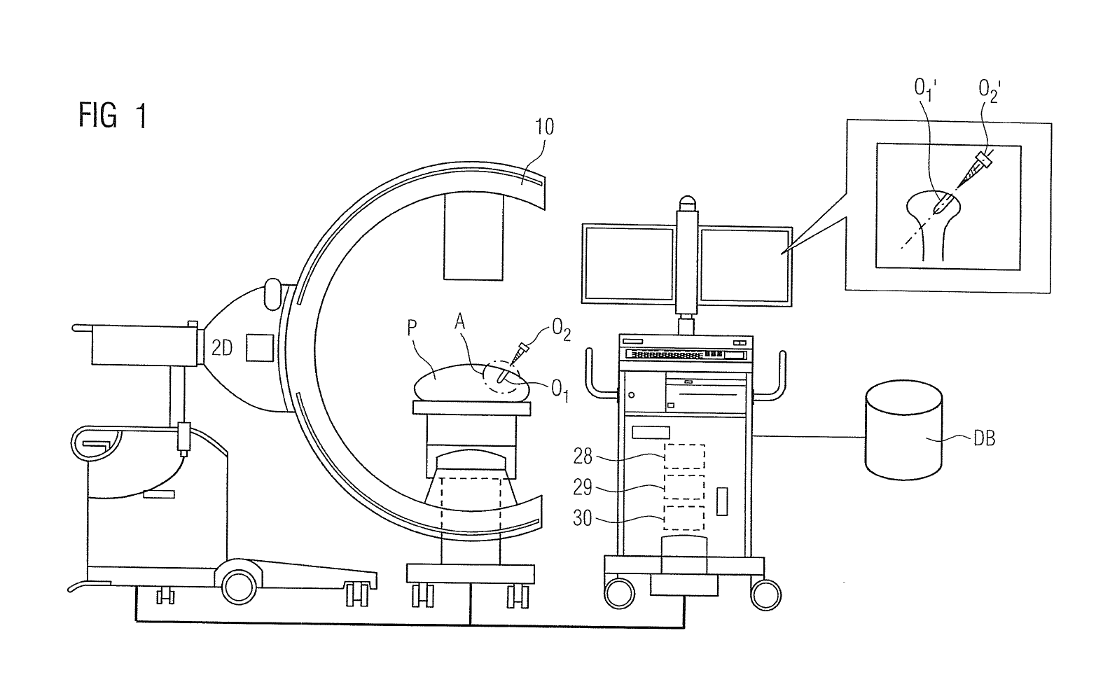

In the following the invention is explained in detail in connection with FIG. 1.

An x-ray apparatus with C-arm to acquire two-dimensional x-ray images should be identified with the reference character 10. The C-arm x-ray apparatus 10 can be fashioned as a mobile apparatus and be borne on wheels for mobile use. The C-arm x-ray apparatus 10 is fashioned with a calculation unit (only schematically indicated in FIG. 1) that is engaged in data exchange with a computer, which computer is likewise borne on a device cart so as to be mobile and comprises a monitor that is fashioned to display the x-ray images. The x-ray apparatus 10 is fashioned to acquire two-dimensional x-ray exposures. It has a patient bed for a patient, an x-ray source and an x-ray detection device. To acquire two-dimensional projections from different projection angles, the C-arm acquiring the x-ray system is displaced by motor in a specific angle range around the patient to be examined or, respectively, around an anatomical structure A of a patient P along the circumference of said C-arm in the direction of a middle longitudinal axis of the patient bed in order to thereby acquire projections of the anatomical structure A.

A computer that has a control and calculation unit to control and operate the C-arm x-ray apparatus 10 is typically housed in the mobile apparatus cart. Moreover, it can comprise units (not shown in FIG. 1) in order to process the acquired image data sets in order to show these on the monitor.

The monitor can be a typical monitor or a double monitor (as shown at the top right in FIG. 1). The monitor is fashioned to show a modified x-ray image.

An essential aspect of the present invention is that three-dimensional information can be visualized in relation to surgical objects O₁, O₂, O₃, . . . Oᵢ, although the x-ray apparatus 10 only acquires two-dimensional image data sets. Examples of surgical objects Oᵢ are implants, instruments and/or tools introduced into the patient body. This is schematically presented in FIG. 1. The patient P is located on the patient bed to implement a medical procedure at the anatomical structure (for example the femur) A. An implant O₁ has already been inserted into the anatomical structure A of the patient P. The implant O₁ should now be attached to the femur with an additional surgical object, namely a tool (for example a screw). The tool is identified with the reference character O₂ in FIG. 1. In a two-dimensional x-ray exposure it is not possible to measure the distance of the tool O₂ from the implant O₁. Moreover, it is also not possible to detect or, respectively, visualize an orientation of the screw O₂ with regard to the implant O₁ since the third dimension is missing in the two-dimensional x-ray image exposure.

According to the invention, two alternatives are provided in order to introduce the three-dimensional information into the two-dimensional x-ray image. A preferred, alternative embodiment of the invention combines the two approaches (model-based and marker-based).

The first approach is model-based, in which a computerized processor accesses a three-dimensional model of each of the relevant surgical objects. The three-dimensional models are typically stored in a database DB. In practice at least two separate surgical objects O₁, O₂ should be presented relative to one another in terms of their spatial position. As mentioned above, these surgical objects are typically an implant O₁ and a tool or instrument O₂ that is to be engaged with the implant O₁. The implant manufacturers normally deliver (provide) a three-dimensional model for their implants that is stored and used according to the invention. Alternatively, the 3D model of the surgical objects can be imported from a remote source via an interface, or can be newly calculated entirely or in part in the event that such a model does not yet exist. The three-dimensional model Oᵢ′ includes all relevant information of the surgical object Oᵢ in three-dimensional space. In a next step, the three-dimensional model Oᵢ′ for the surgical object Oᵢ is integrated into the acquired two-dimensional x-ray image data set. The modified x-ray image that is generated in this manner includes all relevant and integrated three-dimensional models Oᵢ′ in the coordinate system of the C-x-ray arm 10. All relative positions between implant O₁ and tool O₂ can advantageously be measured directly and immediately from the modified x-ray image. Moreover, the orientations of the surgical objects Oᵢ in space can be measured and presented.

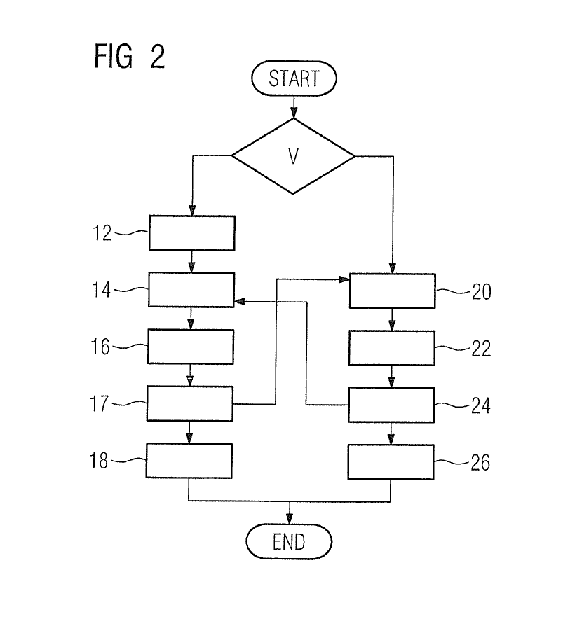

The workflow described in the preceding is summarized again in the left part of FIG. 2. A typical workflow according to the model-based procedure is explained in the following in connection with FIG. 2.

After the start, a selection is offered to the user in order to branch into different branches or workflows. This branching is represented by reference character V in FIG. 2. Depending on the embodiment, a specific workflow is preset or the user can differentiate between the two branches here. In the model-based variant explained in detail above, in Step 12 the two-dimensional x-ray image is acquired by means of the C-arm x-ray apparatus 10. The acquired x-ray image is characterized by at least two surgical objects O₁, O₂ being visible therein. These are normally an implant O₁ and a tool or an instrument O₂ that is yet to be introduced into an anatomical structure A.

An access to the database DB is subsequently executed in Step 14 in that three-dimensional models O₁′, O₂′, O₃′, . . . Oᵢ′ are stored for the respective surgical objects O₁, O₂, O₃, . . . , Oᵢ.

Two-dimensional projections of the three-dimensional models are provided in Step 16. An integration of the provided three-dimensional models Oᵢ′ into the acquired two-dimensional x-ray image can thereupon be executed. This is executed by means of an adaptation calculation. For this purpose, all or a selection of two-dimensional projections of the models in different section axes are provided for the relevant models Oᵢ′ (thus the models that are relevant to the medical procedure). These 2D projections are compared with the acquired two-dimensional x-ray image. In the event that an agreement in the third dimension/axis (thus in the projections) is detected, that 2D projection is selected and integrated as a virtual structure into the acquired x-ray image. A calculation of three-dimensional positions (comprising all relevant position and angle information) of the (real) objects thereby takes place in Step 17.

According to a preferred embodiment of the invention, in a last Step 18 a modified x-ray image is presented on a monitor. The modified x-ray image comprises the integrated three-dimensional models Oᵢ′ in the coordinate system of the imaging apparatus 10, thus a virtual representation of the real objects in three-dimensional form. A basic advantage of the invention is that relevant distances between the real surgical objects O₁, O₂, . . . , Oᵢ and its orientations can be measured and visualized immediately from the presentation of the modified x-ray image.

A second variant of the solution according to the invention is based on a marker-based approach, which second variant can, however, be combined with the first variant according to an alternative embodiment of the invention and in FIG. 2 is shown in the right branch of the workflow model. In a preparation step, x-ray-opaque markers M are thereby attached to the relevant surgical objects O₁, O₂, O₃, . . . . This is identified with the reference character 20 in FIG. 2.

In a next Step 22, a two-dimensional x-ray image is acquired by means of the C-arm x-ray apparatus 10, in which two-dimensional x-ray image the at least two surgical objects O₁, O₂ (or additional objects Oᵢ) or additional objects Oᵢ with the respective markers M are mutually visible.

In a next Step 24, all three-dimensional positions and distances in space between the participating surgical objects O₁, O₂ are measured and presented from the acquired x-ray image with the markers M. The positions and orientations of the surgical objects Oᵢ can be described in the coordinate system of the C-arm. This can also be achieved alternatively or cumulatively by a two-dimensional representation of the three-dimensional model of the surgical object Oᵢ′ being provided. That two-dimensional representation that can be brought into congruence (to the greatest extent possible) with the acquired two-dimensional x-ray image is selected from the set of provided two-dimensional representations. The selected two-dimensional representation of the three-dimensional model Oᵢ′ for the surgical object Oᵢ is then integrated into or overlaid in the acquired x-ray image.

In a last step 26, the modified x-ray image with the integrated three-dimensional data for the surgical objects O₁, O₂ can then be presented in the coordinate system of the x-ray apparatus 10. In both embodiment variants (the model-based and the marker-based embodiment) the modified x-ray image includes all three-dimensional information that is necessary for an exact localization of the relevant surgical objects Oᵢ with their distances relative to one another and with their orientations in space.

In both variants of the invention (marker-based and model-based), 3D positions and 3D orientations of the at least two surgical objects O₁, O₂ are presented in a virtual 3D space. It is thus possible to align a tool or an instrument O₂ (for example a screw or a pin) in relation to its middle longitudinal axis so that the pin or the screw O₂ coincides exactly with the middle longitudinal axis of a sleeve in the implant O₁ into which the pin or, respectively, the screw O₂ should be introduced. This is presented as an example in FIG. 1 with the imaged structures or models O₁′, O₂′.

The method can thus be used for navigation-guided preparation and implementation of a medical procedure and significantly shortens the preoperative treatment time. Furthermore, it is possible to still vary the position and/or the orientation of the tool or of the instrument O₂ outside of the patient P and outside of the anatomical structure A, thus without an interaction with the patient P being necessary. This has previously only been possible in an interaction with the patient.

As shown in FIG. 2, the method typically ends with the presentation 18, 26 of the modified x-ray image. The modified x-ray image is shown on a monitor. This is represented in FIG. 1 in the schematic overview drawing. The modified x-ray image thereby also comprises the three-dimensional models O₁′, O₂′ of the relevant surgical objects O₁, O₂.

As already explained, it is also possible to combine the model-based approach and marker-based approach. This can occur in that the process branches to the model-based method (shown in the left branch in FIG. 1) in Step 17 or to the marker-based workflow in Step 16 (this is represented by the arrow in FIG. 1). In principle it is possible here to branch to Steps 20, 22 or 24 depending on which preparation measures are still required. For reasons of clarity, only a pointer from Step 17 to Step 20 is shown in FIG. 1. Before the calculation in Step 24, or in Step 22, the process can likewise branch from the marker-based method (shown in the right branch in FIG. 1) to the model-based workflow (this is likewise represented as an example by the arrow in FIG. 1 from Step 24 to Step 14). Naturally, other branchings—and therefore a different order—for the execution of the method steps are also within the scope of the invention here.

The method described in detail in the preceding can be implemented by software modules and/or hardware modules in a microprocessor.

In addition to the x-ray apparatus 10 and the database DB to store the three-dimensional models Oᵢ′, the system according to the invention comprises a calculation module 28 (possibly an integration module 29) that serves to mathematically adapt or match the 3D model data in the acquired 2D x-ray image, as well as a presentation module 30 that serves to show the modified x-ray image. Depending on the embodiment of the present invention, the calculation module 28 is fashioned in order to execute the model-based approach and/or the marker-based approach or a combination of both, as described in detail above.

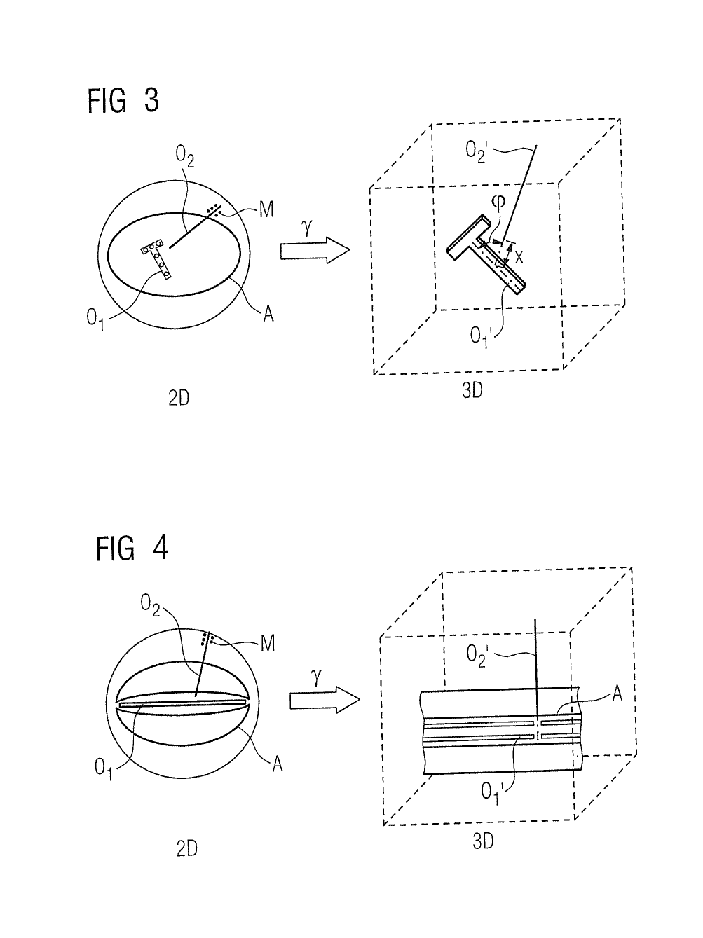

In the following the basis of the present invention is again be explained in detail using two examples in connection with FIGS. 3 and 4.

FIG. 3 concerns an application example in which an implant O₁ should be temporarily fixed with a k-wire O₂. In this way the k-wire O₂ can be brought exactly into the correct position with the assistance of the three-dimensional representation and the two-dimensional x-ray imaging, without x-ray exposures in addition to the one acquired x-ray image being necessary. In the example shown in FIG. 3, the k-wire is provided with one or more markers M in order to obtain three-dimensional information. According to the invention, a virtual presentation is calculated from these data (acquired two-dimensional x-ray image, acquired three-dimensional model O₁′ of the implant O₁ and acquired three-dimensional model O₂′ of the k-wire O₂ and/or marker-related data), wherein the position data are transformed. This transformation is identified with the reference character γ in FIGS. 3 and 4. The transformation serves to show all relevant three-dimensional information in the 3D space of the coordinate system of the C-arm, which should be shown in FIG. 3 on the right side in the form of an indicated cube. The modified x-ray image is thus shown on the right side with the three-dimensional models O₁′ and O₂′. In the event that it is desired, the distance between the k-wire O₂ and the implant O₁ can additionally be shown here. It is likewise possible to indicate another angle range that exists between the k-wire O₂ and the implant O₁. In addition to this information, additional data can be indicated. For example, additional assistance can be shown here for navigation by the surgeon, for instance in the form of warning notifications in the event that the k-wire O₂ to be integrated has an incorrect distance or an incorrect orientation with regard to the implant O₁.

The example shown in FIG. 4 concerns a distal pin locking at an anatomical structure A (for example a bone). According to the invention it is possible that the k-wire O₂ can be guided immediately and directly to its target to lock the pin O₁. As is schematically shown on the left side in FIG. 4, the acquired x-ray image comprises the anatomical structure A (in this case the bone) with the already-introduced pin O₁ and a k-wire O₂ that, in this example, is provided with marker M. After the transformation γ in the 3D space of the coordinate system of the C-arm 10, the relevant three-dimensional information can then be presented in the form of three-dimensional models O₁′ and O₂′. This is shown on the right side in FIG. 4. The k-wire O₂ can thus be navigated or, respectively, positioned exactly at the engagement point with the pin O₁ without it being necessary to execute a new x-ray acquisition of the patient.

Individual modules explained herein are to be understood as software and hardware modules, and the exemplary embodiments are to be understood as not limited with regard to a specific physical realization of the invention. In particular, for those skilled in the art it is apparent that the invention can be realized in software and/or hardware and/or partially or completely distributed at multiple physical products, in particular computer program products as well.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for three-dimensional presentation of at least two separate surgical objects in an image-assisted medical procedure, comprising:
with an imaging apparatus, acquiring a two-dimensional x-ray image in which at least two surgical objects are both shown;
from a computerized processor, accessing a three-dimensional model in electronic form respectively for each of said at least two surgical objects;
in said computerized processor, for each three-dimensional model, determining a plurality of two-dimensional projections of the three-dimensional model and selecting the two-dimensional projection, from among said plurality of two-dimensional projections, that is best adapted to the showing of the respective surgical object in the two-dimensional x-ray image;
in said computerized processor, calculating, for each of said at least two surgical objects, a three-dimensional position thereof for the selected best-adapted two-dimensional projection thereof; and
from said computerized processor, displaying the respective calculated three-dimensional positions of each of said at least two surgical objects in at least one of a virtual three-dimensional space and a modified x-ray image.

2. A method as claimed in claim 1 comprising selecting said surgical objects from the group consisting of surgical tools, surgical instruments, and medical implants.

3. A method as claimed in claim 1 comprising, for each of said surgical objects, selecting the best-adapted two-dimensional projection by an image registration procedure comprising a coordinate transformation, selected from the group consisting of two-dimensional image registration procedures and three-dimensional image registration procedures.

4. A method as claimed in claim 1 wherein said calculated three-dimensional positions of each of said at least two surgical objects are displayed in a modified x-ray image, and comprising selecting said modified x-ray image as a virtual presentation of said at least two surgical objects and a plurality of slice images.

5. A method as claimed in claim 1 comprising, in said display, overlaying at least one item selected from the group consisting of information other than said surgical objects, method data, and three-dimensional representations of items other than said three-dimensional objects.

6. A method as claimed in claim 1 comprising generating said display as an image fusion with an additional image.

7. A method as claimed in claim 1 comprising generating said display to include data for navigation of at least one of said surgical objects in a form allowing navigation-guidance of said medical procedure.

8. A method as claimed in claim 1 comprising generating said display repeatedly or continuously during said medical procedure and updating said display when a change selected from the group consisting of a relative position and a relative orientation is detected between said at least two surgical objects.

9. A method as claimed in claim 8 comprising generating and updating said display in real time.

10. A method as claimed in claim 1 wherein said display of the calculated three-dimensional positions of said at least two surgical objects is said modified x-ray image, and comprising localizing said at least two surgical objects in a coordinate system of the imaging apparatus using said modified x-ray image.

11. A method as claimed in claim 1 comprising acquiring said two-dimensional x-ray image pre-operatively, before beginning said medical procedure.

12. A method as claimed in claim 1 comprising acquiring said two-dimensional x-ray image at respective points in time during said medical procedure, selected from the group consisting of predetermined points in time and points in time respectively triggered by events occurring in said medical procedure.

13. A method as claimed in claim 1 comprising automatically detecting a change in a relative position or angle between said at least two surgical objects and, upon detecting said change, automatically acquiring said two-dimensional x-ray image and implementing said calculations in said computerized processor to generate said display.

14. A method for three-dimensional presentation of at least two separate surgical objects in an image-assisted medical procedure, comprising:
attaching a plurality of x-ray-opaque markers with a known geometry to each of at least two surgical objects;
with an imaging apparatus, acquiring a two-dimensional x-ray image in which at least two surgical objects are both shown;
from a computerized processor, accessing a three-dimensional model in electronic form respectively for each of said at least two surgical objects;
in said computerized processor, for each three-dimensional model, determining a plurality of two-dimensional projections of the three-dimensional model and selecting the two-dimensional projection, from among said plurality of two-dimensional projections, that is best adapted to the showing of the respective surgical object in the two-dimensional x-ray image;
in said computerized processor, calculating, for each of said at least two surgical objects, a three-dimensional position thereof for the selected best-adapted two-dimensional projection thereof; and
from said computerized processor, displaying the respective calculated three-dimensional positions of each of said at least two surgical objects in at least one of a virtual three-dimensional space and a modified x-ray image.

15. A method for three-dimensional presentation of at least two separate surgical objects in an image-assisted medical procedure, comprising:
attaching a plurality of x-ray-opaque markers with a known geometry to each of at least two surgical objects;
with an imaging apparatus, acquiring a two-dimensional x-ray image in which said at least two surgical objects, with said markers respectively attached thereto, are both shown;
in a computerized processor, calculating, from said markers for each of said at least two surgical objects shown in said two-dimensional x-ray image, three-dimensional positions in space between the at least two surgical objects; and
from said computerized processor, displaying the respective calculated three-dimensional positions of each of said at least two surgical objects in at least one of a virtual three-dimensional space and a modified x-ray image.

16. A system for three-dimensional presentation of at least two separate surgical objects in an image-assisted medical procedure, comprising:
an imaging apparatus that acquires a two-dimensional x-ray image in which at least two surgical objects are both shown;
a computerized processor configured to access a three-dimensional model in electronic form respectively for each of said at least two surgical objects;
said computerized processor being configured to determine, for each three-dimensional model, a plurality of two-dimensional projections of the three-dimensional model and to select the two-dimensional projection, from among said plurality of two-dimensional projections, that is best adapted to the showing of the respective surgical object in the two-dimensional x-ray image;
said computerized processor being configured to calculate, for each of said at least two surgical objects, a three-dimensional position thereof for the selected best-adapted two-dimensional projection thereof; and
said computerized processor being configured to display the respective calculated three-dimensional positions of each of said at least two surgical objects in at least one of a virtual three-dimensional space and a modified x-ray image.

17. A system for three-dimensional presentation of at least two separate surgical objects in an image-assisted medical procedure, comprising:
a plurality of x-ray-opaque markers with a known geometry to each of at least two surgical objects;
an imaging apparatus that acquires a two-dimensional x-ray image in which said at least two surgical objects, with said markers respectively attached thereto, are both shown;
a computerized processor configured to calculate, from said markers for each of said at least two surgical objects shown in said two-dimensional x-ray image, three-dimensional positions in space between the at least two surgical objects; and
said computerized processor being configured to display the respective calculated three-dimensional positions of each of said at least two surgical objects in at least one of a virtual three-dimensional space and a modified x-ray image.

18. A computerized processor for three-dimensional presentation of at least two separate surgical objects in an image-assisted medical procedure, comprising:
a processor input supplied with a two-dimensional x-ray image in which at least two surgical objects are both shown;
a calculation module configured to access a three-dimensional model in electronic form respectively for each of said at least two surgical objects;
said calculation module being configured to determine, for each three-dimensional model, a plurality of two-dimensional projections of the three-dimensional model and to select the two-dimensional projection, from among said plurality of two-dimensional projections, that is best adapted to the showing of the respective surgical object in the two-dimensional x-ray image;
said calculation module being configured to calculate, for each of said at least two surgical objects, a three-dimensional position thereof for the selected best-adapted two-dimensional projection thereof;
a processor output; and
said computation module being configured to produce and emit a data file, at said processor output, in a form that allows displaying the respective calculated three-dimensional positions of each of said at least two surgical objects in at least one of a virtual three-dimensional space and a modified x-ray image.

19. A computerized processor for three-dimensional presentation of at least two separate surgical objects in an image-assisted medical procedure, comprising:
a processor input supplied with a two-dimensional x-ray image in which at least two surgical objects are both shown with a plurality of x-ray-opaque markers with a known geometry attached to each of at least two surgical objects;
a computation module configured to calculate, from said markers for each of said at least two surgical objects shown in said two-dimensional x-ray image, three-dimensional positions in space between the at least two surgical objects; and
said computation module being configured to produce and emit a data file, at said processor output, in a form that allows displaying the respective calculated three-dimensional positions of each of said at least two surgical objects in at least one of a virtual three-dimensional space and a modified x-ray image.

20. A non-transitory computer-readable storage medium encoded with programming instructions, said storage medium being loadable into a computerized processor and causing said computerized processor to:
receive a two-dimensional x-ray image in which at least two surgical objects are both shown;
access a three-dimensional model in electronic form respectively for each of said at least two surgical objects;
for each three-dimensional model, determine a plurality of two-dimensional projections of the three-dimensional model and select the two-dimensional projection, from among said plurality of two-dimensional projections, that is best adapted to the showing of the respective surgical object in the two-dimensional x-ray image;
calculate, for each of said at least two surgical objects, a three-dimensional position thereof for the selected best-adapted two-dimensional projection thereof; and
cause the respective calculated three-dimensional positions of each of said at least two surgical objects to be displayed in at least one of a virtual three-dimensional space and a modified x-ray image.

21. A non-transitory computer-readable storage medium encoded with programming instructions, said storage medium being loadable into a computerized processor and causing said computerized processor to:
receive a two-dimensional x-ray image in which at least two surgical objects are both shown with a plurality of x-ray-opaque markers with a known geometry attached to each of at least two surgical objects;
calculate, from said markers for each of said at least two surgical objects shown in said two-dimensional x-ray image, three-dimensional positions in space between the at least two surgical objects; and
cause the respective calculated three-dimensional positions of each of said at least two surgical objects to be displayed in at least one of a virtual three-dimensional space and a modified x-ray image.
